(19) United States
(12) Patent Application Publication
(10) Pub. No.: US 2006/0069284 A1
(43) Pub. Date: Mar. 30, 2006

(54) Esters of allyl substituted by a difluoromethylene group, method for the synthesis thereof and use thereof

(75) Inventors: Nicolas Roques, Gaillac (FR); Laurent Saint James, Vourles (FR); Jean-Marie Bernard, Saint Laurent D'Agny (FR)

Correspondence Address: Jean-Louis Seugnet;Rhodia Inc, Intellectuual Property Dept, 259 Prospect Plains Road CN-7500, Cranbury, NJ, 08512, US

(73) Assignee: RHODIA CHIMIE, 92512 BOULOGNE BILLANCOURT CEDEX (FR)

(21) Appl. No.: 10/539,639

(22) PCT Filed: Dec. 17, 2003
(86) PCT No.: PCT/FR03/03780

§ 371 Date: Jun. 16, 2005

(30) Foreign Application Priority Data
Dec. 20, 2002 (FR) 02/16308

(51) Int. Cl. C07C 67/02

(52) U.S. Cl. 560/262

(57) ABSTRACT

The subject matter of the present invention is compounds forming an allyl ester in which the allyl group is substituted by a difluoromethylene group; another subject matter of the invention is a route for the synthesis of these compounds and their use in cycloaddition processes for the synthesis of nitrogenous heterocycles, in particular 3+2 cycloadditions.

The subject matter of the present invention is compounds forming an allyl ester in which the allyl group is substituted by a difluoromethylene group.

Another subject matter of the invention is a route for the synthesis of these compounds and their use in cycloaddition processes, in particular 3+2 cycloadditions.

During the last decade, compounds carrying fluoroalkylated groups have become much more widely used, in particular in agrochemistry and pharmaceutical products.

In particular, heterocycles, especially nitrogenous heterocycles and especially 5-membered heterocycles, have become relatively common when they carry —CF₂— groups. However, the synthesis of such compounds is difficult and often requires a lengthy sequence of stages.

Consequently, one of the aims of the present invention is to provide a family of precursors of these nitrogenous heterocycles. The use of these precursors has to be easy and must not require numerous stages.

Another aim of the present invention is to provide a process for the synthesis of said precursors.

Another aim of the present invention is to provide a technique for the use of these precursors.

These aims, and others which will become apparent subsequently, are achieved by means of compounds of formula (I): 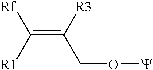
in which:
R_{f} is a radical carrying a perfluoromethylene group, which group provides the link with the remainder of the molecule;
R₁ and R₃, which can be the same or different, are chosen from hydrogen and alkyl or aryl radicals;
is an electron-withdrawing group such that Ψ-O—H is an acid with a pKa value (in water) of at most 8, advantageously of at most 6, preferably of at most 5.

According to the present invention, it is desirable for the Ψ-O—H group to constitute an acid with a pKa value of at least 1, advantageously of at least 2. This is because it is desirable for the Ψ-O⁻ group not to constitute a good leaving group, this being the case both for the use as cycloaddition precursor and for its synthesis.

It is desirable for R₁ and R₃ not to excessively hinder the molecule; it is consequently advisable to prevent R₁ and/or R₃ from being attached to the double bond via a tertiary, indeed even secondary, carbon.

Thus, it is preferable for at least one of the R₁ and R₃ groups to be a light alkyl (light, that is to say of at most 4 carbons) or better still a hydrogen.

Thus, it is preferable for R₁ to be hydrogen; it is also preferable for R₃ to be hydrogen; and it is even more preferable for R₁ and R₃ to be hydrogens.

Advantageously, R_{f} is of formula (II)
EWG-(CX₂)ₚ—
where:
the X units, which are alike (that is to say that they are identical) or different, represent a chlorine, a fluorine or a radical of the formula CₙF₂ₙ₊₁ with n an integer at most equal to 5, preferably at most equal to 2, with the condition that the X units of the methylene group carrying the open bond are not chlorine and that one of them at least is a fluorine atom; advantageously the X units of the methylene carrying the open bond are either fluorine atoms or else a fluorine atom and a radical of the formula CₙF₂ₙ₊₁ (this is because such radicals are regarded as being similar electronically to fluorine atoms),
   EWG is a hydrocarbon or electron-withdrawing group (that is to say that the Hammett constant σₚ (sigma p) is >0, advantageously at least equal to 0.2) which is preferably inert and advantageously, when p is equal to 1, is an electron-withdrawing group (cf. preceding lines);
   p is a positive integer, that is to say that it cannot comprise the value 0.

EWG is advantageously fluorine, in particular when p is less than or equal to 2.

The X units are advantageously all fluorines, in particular when p is less than or equal to 2.

Another value of EWG (electron-withdrawing group) is the value chlorine; in this case, EWG is a chlorine.

p represents an integer, advantageously at most equal to 4, preferably at most equal to 2.

EWG advantageously represents an electron-withdrawing group, the possible functional groups of which are inert under the conditions of the reaction, advantageously fluorine or a perfluorinated residue of formula CₙF₂ₙ₊₁ with n an integer at most equal to 8, advantageously at most equal to 5.

The total number of carbons of R_{f} is advantageously between 1 and 15, preferably between 1 and 10.

It is advantageous for R_{f} to be of formula CᵣF₂ᵣ₊₁ with r a positive integer ranging from 1 to 10, advantageously from 1 to 5, preferably from 1 to 3.

The present invention is particularly advantageous for the R_{f} groups of low molecular weight, that is to say, those which are relatively volatile (that is to say, corresponding to an R_{f}H with a boiling point at atmospheric pressure at most equal to 100° C.). The technique is particularly advantageous for the R_{f} groups having a radical exhibiting an odd number of carbons and particular mention must be made of the R_{f} groups with 1, 2 and 3 carbons. Thus, the trifluoromethyl, pentafluoroethyl and heptafluoropropyl radicals number among the preferred values of the R_{f} group.

According to the present invention, it is preferable for Ψ to represent an acyl, advantageously such that the pKa value (measured or converted to a value in water) is at least equal to approximately 2 (the expression “approximately” is used here to emphasize that the number which follows it corresponds to a mathematical rounding off), advantageously to approximately 3.

Ψ-O—H is usually an alkanoic acid, advantageously of 1 to 8 carbon atoms, preferably of 2 to 5 carbon atoms.

In economic terms, the value of Ψ equal to acetyl is the most advantageous.

As was mentioned above, another aim of the present invention is to provide an easy route of access to the molecules mentioned above.

This aim is achieved by means of a process for the synthesis of the compound of the formula (I) which comprises bringing a compound of formula (III): 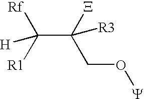
(where Ξ (xi) represents a halogen (heavier than fluorine, av chlorine or bromine) or pseudohalogen leaving group such that ΞH exhibits a Hammett constant at least equal to that of trifluoroacetic acid, advantageously at least equal to that of mesylic acid) into contact with a base chosen from strong nitrogenous bases, the associated acid of which exhibits a pKa value of at least 12, and/or from anionic bases, with the condition that, when the base is a nonnitrogenous anionic base, the latter is in the presence of a polar solvent or a mixture of polar solvents.

To date, access to the compounds of formula (I) has not been described; a good reason for this is that the most direct route starting from known compounds is difficult, is arduous and, generally, results in products other than that desired (see in particular the comparative examples).

The compounds of formula (III) are delicate compounds, the purification of which is often difficult, indeed even virtually impossible, due to the instability, in particular thermal instability, of the compounds of formula (III), in particular when Ξ represents a halogen forming a good leaving group, such as bromine or iodine.

The chlorine derivative is somewhat more stable. The technique for the synthesis of the compounds of formula (III) has been disclosed in International Application PCT WO 01/58833, filed on behalf of the Applicant Company.

Generally, the synthesis of the compounds of formula (III) can be carried out by the reaction defined by the equation below: 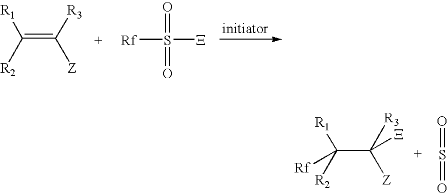
with Z representing a methylene group (optionally substituted but preferably unsubstituted) carrying Ψ-O— and Ξ limited here to Cl and Br, indeed even to I (but the sulfonyl iodide is unstable; its existence, which might only be transitory, has not been demonstrated and it cannot be employed, unless it is prepared in situ).

The detachment of the acid HΞ from the molecule of formula (III) to give the molecule of formula (I) is difficult to control, the majority of bases giving nucleophilic substitution or decomposition reactions, indeed even reactions for the elimination of a halogen from the R_{f} group.

Following the study which has led to the present invention, it was shown that, by choosing certain bases, the reaction could take place in the desired direction. It has also been shown that other bases could be used, provided that the operating conditions and in particular the reaction medium were chosen.

It can thus be indicated that the bases below can give good results:
nonanionic bases in which a nitrogen doublet is conjugated with a carbon-nitrogen double bond; these bases can either be used alone, in stoichiometric or excess stoichiometric amounts, or can be used in catalytic amounts with another base, preferably more basic than the said nonanionic bases; in particular, these bases can be used with the bases below, which are in principle used in stoichiometric or excess stoichiometric amounts;
nitrogenous anionic bases, which give good results but are generally expensive and the effect of which improves in proportion as polar aprotic solvents are used as solvents;
nonnitrogenous anionic bases, advantageously not resulting from an alcohol or from water (hydroxide and alkoxide ions are respectively to be banned and, preferably, to be avoided). These bases only give good results when a polar solvent, the donor number of which is at least equal to 10, advantageously to 15, preferably to 20, is used.

When polar solvents are used according to the process of the invention, it is preferable for these polar solvents to exhibit a dielectric constant ε (epsilon) of at least 7. Furthermore, when the solvent is basic, that is to say when it exhibits a high donor number (greater than 20), it is preferable for this basicity to be relatively low within the meaning of Bronsted basicity, that it to say for the pKa of the associated acid of said solvent to be greater than 5, advantageously greater than 6, more preferably greater than 7.

Advantageously, said reaction medium is aprotic and anhydrous. In particular, it is desirable for this aprotic and anhydrous medium to be such that the strongest acid present in the medium, not taking into account the substrate, exhibits a pKa value of at least 20, better still of at least 25, advantageously of at least 30, preferably of at least 35. This constraint is targeted at preventing side reactions during the detachment of the proton from the substrate by the base; this is because the anions resulting from the detachment of a proton by a base are nucleophiles which can result in a nucleophilic substitution reaction, which is not desired.

Thus, it is preferable for such anions not to be formed. More generally, it is preferable for the constituents of the reaction mixture not to be, on contact with the base used, capable of giving nucleophilic anions. Strong acids (pKa≦2), indeed even moderate acids (2<pKa≦4.5), do not interfere strictly speaking with the reaction as they consume base, giving only anions of little or no nucleophilicity and therefore only little or nothing in the way of side reaction(s). The acids associated with the bases according to the present invention, or resulting from them, obviously are not harmful to the present invention. This is the reason why hydroxide ions, indeed even alkoxide ions, are not suitable for the processes according to the present invention. This is because they result in molecules which are to be avoided as solvents.

Thus, it is preferable for the content of labile hydrogen atoms (that is to say, those not corresponding to the specific pKa values above) in the reactant to be at most equal to ⅓, advantageously to ¼, preferably to 10% (in moles), with respect to the initial content of that of said base or of said compound of formula (III) which is not in excess.

One of the advantages of cryptands is that they make it possible to dispense, at least partially, with solvents with a high donor number.

For the definition of “donor number”, reference may be made to the work by Christian Reichardt, Solvents and Solvent Effects in Organic Chemistry, p. 19 (1988), in which work it is found defined as the negative of the enthalpy (—ΔH, expressed in kilocalorie/mole) of the interaction of the solvent with antimony pentachloride in a dilute dichloromethane solution.

In the case of mixtures of solvents or of solvents comprising cryptands, the donor number will be calculated by donor function, by multiplying the donor number of each of the solvents by the molar fraction which it represents and by summing these products.

Mention may be made, among nitrogenous anionic bases, of salts, in particular alkali metal or alkaline earth metal salts, of silylated or nonsilylated amines and of silylamines. Mention may be made, among the salts giving the best results, of salified disilylamines and in particular the salts, especially alkali metal or alkaline earth metal salts, of hexamethyldisilazane (HMDZ).

Mention may be made, among nonnitrogenous anionic bases, of non-oxygen-comprising bases, such as, for example, alkali metal or alkaline earth metal hydrides and alkane salts, such as butyllithium, and alkali metal carbonates. It should be remembered that the bases of this family have to be used in the presence of polar solvents.

It should be remembered that it is desirable to use readily distillable solvents (B.p. less than or equal to 120° C.) and/or solvents which are miscible with water, advantageously in any proportion, in order to more easily treat the reaction mixture.

The bases giving the best results are the bases comprising 2 conjugated nitrogen atoms, as has already been mentioned and as is described in detail below.

According to an advantageous embodiment according to the present invention, said base is nitrogenous and nonanionic and corresponds to the formula (IV):
D—A″═A—R2
where A is a semimetallic atom from Group VB (group comprising nitrogen and advantageously the latter) (the Periodic Table of the Elements used in the present application is that of the Supplement to the Bulletin de la Société Chimique de France, January 1966, No. 1),
where A″ is a carbon atom carrying hydrogen or substituted by a hydrocarbon radical R₅,
where the radical D is:
   either a doublet-carrying semimetal chosen from:
      chalcogens advantageously monosubstituted by a monovalent radical R₆ (in which case the chalcogens constitute said doublet-carrying semimetal),
      semimetals of Group VB, in particular nitrogen or phosphorus (in which case the semimetals of Group V constitute said doublet-carrying semimetal), preferably nitrogen, which semimetals of Group VB are advantageously disubstituted by two monovalent hydrocarbon radicals R₆ and R′₆ to form a radical D of formula —A′(R₆) (R′₆);
   or a radical carrying both a semimetal atom and at least one unsaturation, said unsaturation or unsaturations providing the conjugation between a doublet of said semimetal atom and the —A3═A— double bond; this group is connected to the remainder of the molecule via a single bond carried by an atom chosen from carbon atoms of sp² hybridization substituted by a functional group or a divalent radical R₇, carrying a hydrogen or optionally substituted by a hydrocarbon radical R₆.

It should be remembered that, in this formula, the semimetals of Group VB are preferably a nitrogen, whether this is for A″ or for A′.

When A″ is an atom of Group VB, and in particular a nitrogen, it is preferable for D to be chosen from those described above in which the single bond providing the connection with the remainder of the molecule is carried by an atom chosen from carbon atoms of sp² hybridization substituted by a functional group or by a divalent radical R₇, carrying a hydrogen or optionally substituted by a hydrocarbon radical R₆, to give a formula for D specified below:

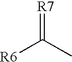

When said carbon carries a hydrogen, this hydrogen is found in place of R₆ to thus give the value hydrogen to R₆.

As has been said above, it is desirable for the base of formula (IV) to comprise a semimetal atom (saturated, that is to say not carrying a double bond) exhibiting a resonance (or conjugation) with a π bond connecting two atoms, at least one of which is a disubstituted and positively charged atom of Group VB; advantageously an organic base comprising a trivalent atom of Group VB (nitrogen column in the Periodic Table), advantageously nitrogen, the doublet of which atom is conjugated directly or indirectly with a π bond connecting two atoms, at least one of which is an atom of Group VB (namely A).

According to a particularly advantageous use of the present invention, said π bond connecting two atoms is the π bond of an imine functional group (>C═N—).

This imine functional group can be written in the following way: 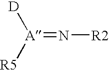
with A″ representing a carbon,
with D chosen from:
   chalcogens monosubstituted by a monovalent radical R₆,
   a semimetal of Group VB, nitrogen advantageously substituted by two monovalent radicals R₆ and R′₆ in particular or phosphorus, preferably nitrogen; and
   those described above or the connection with the remainder of the molecule is provided by a bond carried by sp² carbon atoms substituted by a functional group or a divalent radical R₇, carrying a hydrogen or optionally substituted by a hydrocarbon radical R₆.

The radical R₅ is chosen from hydrogen, the values of D and hydrocarbon radicals, advantageously aryl radicals and in particular alkyl radicals.

It is preferable for the radical D and this imine functional group to be positioned in such a way that the atoms of nitrogen and of said semimetal are as far apart as possible, in other words, and for example, for the nitrogen of the imine functional group to be that of the two atoms connected via the π bond which is the more distant from the trivalent atom of Group V. That which has just been said with respect to the imine functional group is general for all the atoms of Group VB connected via the π bond in the case where the π bond comprises a carbon atom and an atom of Group V.

According to the present invention, it is preferable for the organic cation comprising a trivalent atom of Group VB, the doublet of which is conjugated with a π bond, to exhibit a sequence, or rather a backbone, of formula >N—[C═C]_{ν}—C═N⁺< with ν equal to zero or an integer chosen within the closed range (that is to say, comprising the limits) 1 to 4, advantageously from 1 to 3, preferably from 1 to 2. Preferably, the preceding sequence corresponds to the formula:
Q—[C(R₈)═C(R₆)]_{ν}—C(R₅)═N (R₂)
with Q representing
   a chalcogen substituted by an aliphatic or aromatic radical R₉; or
   a phosphorus disubstituted or more preferably a nitrogen disubstituted by two identical or different aliphatic or aromatic radicals R₉ and R₁₀: (R₁₀) (R₉)N—;
with ν equal to zero or an integer chosen within the closed range (that is to say, comprising the limits) 1 to 4, advantageously from 1 to 3, preferably from 1 to 2, and where R₂ is chosen from hydrocarbon derivatives, advantageously alkyl derivatives, of at most 4 carbon atoms and hydrogen.

Advantageously, according to the present invention, said trivalent atom of Group VB forms or constitutes a tertiary amine.

More specifically, it is desirable for said organic base comprising a trivalent atom of Group VB, the doublet of which is conjugated with a π bond, to constitute a molecule of the following formula:
(R₁₀) (R₉)N—[C(R₈)═C(R₆)]_{ν}—C(R₅)═N—(R₂)
with ν equal to zero or an integer chosen within the closed range (that is to say, comprising the limits) 1 to 4, advantageously from 1 to 3, preferably from 1 to 2, and where R₂, R₅, R₆ and R₈, which are identical or different, are chosen from hydrocarbon groups, advantageously alkyl groups, of at most 4 carbon atoms and hydrogen and where R₁₀ and R₉, which are identical or different, are chosen from hydrocarbon groups, advantageously alkyl groups, of at most 4 carbon atoms, it being possible for one or two of the R₂, R₅, R₆, R₈, R₉ and R₁₀ substituents to be connected to other remaining substituent(s) to form one, two or more rings, in particular aromatic rings; see below.

The potentializing effect of the base is particularly marked when said π bond connecting two atoms is intracyclic (or when a mesomeric form is intracyclic), even when it is intracyclic in an aromatic ring.

This is in particular the case with pyridine rings, diazine rings (preferably meta-diazine rings, see formulae below) and rings which are derived therefrom, such as quinoline or isoquinoline, such as, for example:

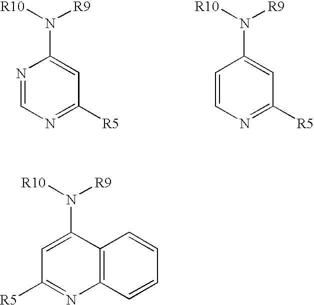

More specifically, the organic base comprising a saturated semimetal atom exhibiting a resonance with a π bond can advantageously be chosen from dialkylaminopyridines, in particular in the para- or ortho-position (that is to say, in the 2- or 4-position of the pyridine; see formula above). Thus, said base carrying at least 2 trivalent nitrogens is advantageously such that said 2 trivalent nitrogens form a bonding system comprising an imine conjugated with the doublet of an amine. Amines which form, with the imine functional group, a substituted amidine functional group which is advantageously intracyclic with 1 ring, indeed even 2 rings, such as DBU (DiazaBicycloUndecene, which has 9 carbon atoms) or DBN (DiazaBicycloNonene, which has 7 carbon atoms), also constitute particularly advantageous bases for use in the present invention. Examples of such bases are to be found in diazabicycloalkenes comprising 6 to 15 carbon atoms:

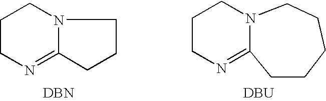

The five-membered rings are also advantageous when they have two or three heteroatoms, for example structures of imidazole, oxazole or cyclic guanidine type, indeed even indole type:

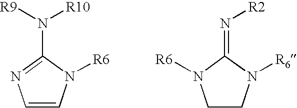

R₆′ and R₆″ having the same value as R₆.

It is possible to substitute the free aryl ring members (participating in an aromatic) or free aliphatic ring members (the point of attachment of which is an sp³ carbon). However, there is not very much advantage to doing this and it has the disadvantage of making the base bulkier.

Triazole structures can also be envisaged:

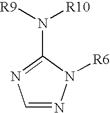

Pyrazole structures are also possible.

It should also be mentioned that, among noncyclic structures, there may be a certain advantage in using guanidine structures, which exhibit the characteristic of being readily derived from guanidine and of exhibiting a formula possessing high resonance: 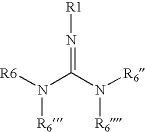
where R₆″′ and R₆″″ are chosen from the same values as R₆; they can be identical to or different from the other R₆ groups and from the R₂ groups. It is preferable, if compounds with a low melting point are desired, for the molecule to be asymmetric. R₆″′ and R₆″″ can be connected to one another to form rings, advantageously aromatic rings.

It is desirable for the molecular mass of the base to be at most equal to 300, advantageously to 250, more preferably to 200. When the bases are polyfunctional (that is to say, carry several basic systems as described above), these values are to be with respect to each basic functional group D—A″═A—R₂.

Another aim of the present invention is to find a cycloaddition technique capable of being used with the compounds of formula (I).

During the study which led to the present invention, it was possible to demonstrate that, provided conditions were relatively mild, that is to say a temperature of less than 150° C., preferably at most equal to 100° C., cycloadditions or cyclocondensations with a cosubstrate carrying 2 double bonds give good results and that their use gives rings, in particular heterocycles substituted by an R_{f} group.

Although it is possible to carry out cycloadditions of 2+4 type, one of the main advantages of the present invention is that of providing ready access to heterocycles by an addition of 3+2 type and in particular 1,3-dipolar cycloadditions. Advantageously, the cosubstrate is an organic compound carrying a pentavalent nitrogen itself carrying 2 double bonds (including bonds of donor-acceptor type), at least one double bond of which connects said nitrogen to a carbon. In particular, the cosubstrates providing the part composed of 3 atoms correspond to the dipolar canonical forms below:

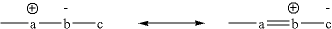

In this instance, b can be an optionally substituted nitrogen and a and c can be oxygen, nitrogen or carbon, it being possible for the two latter atoms to carry a hydrocarbon radical or a hydrogen.

For this type of reaction, reference is made to the general work Advanced Organic Chemistry, third edition, by Jerry March, page 743 et seq., and to the documents cited in this reference work.

The heating of these compounds gives cycloaddition in the presence or absence of a solvent, even without catalyst. However, some of the compounds with a canonical form which has been mentioned above have to be synthesized in situ.

The following nonlimiting examples illustrate the invention.

### EXAMPLE 1

### Dehydrohalogenation of the Precursor CF₃CH₂CHClCH₂OAc

In this instance, R_{f} is trifluoromethyl, R₁ and R₃ are hydrogen and Ξ is chlorine.

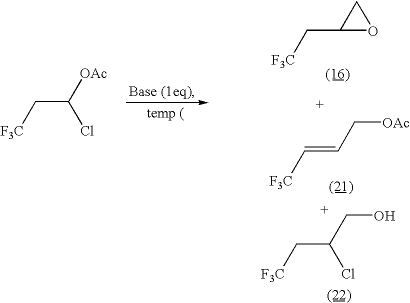

The reaction equation is given above with some of the impurities identified.

| Bases (pKa | | | | | | | | | | |
|---|---|---|---|---|---|---|---|---|---|---|
| of the | | | | | | | | | | |
| associated | (3a) | Base | | t | | DC^{(a)} | RY^{(a)} | RY^{(a)} | RY^{(a)} | |
| acid) | mmol | eq. | Solvents | (h) | Temp. (° C.) | (3a) % | (16) % | (21) % | (22) % | CY % |
| DBU (12) | 1.1 | 1 | Diisopropyl ether | 17 | 0^{(b)} at a.t. | 59 | 0 | 49.1 | 0 | 83.22 |
| | | | DN ˜18 | | | | | | | |
| DBU (12) | 1.1 | 1 | Diisopropyl ether | 4 | 50 | 69.2 | 0 | 71.2 | 0 | 102.89 |
| DBU (12) | 1.1 | 1 | Diisopropyl ether | 4 | 70 | 91 | 0 | 77.5 | 0 | 85.16 |
| Et₃N (10.8) | 1.1 | 1 | Diisopropyl ether | 17 | 20 | 14.5 | 0 | 0 | 0 | 0.00 |
| Et₃N | 1.1 | 1 | Diisopropyl ether | 4 | 50 | 5.8 | 0 | 0 | 0 | 0.00 |
| DABCO | 1.1 | 1 | Diisopropyl ether | 4 | 50 | 4.75 | 0 | traces | 0 | 12.63 |
| NaH (31) | 2.44 | 0.9 | PhCl | 4 | 0^{(c)} at a.t. | 41.1 | 2.2 | 0 | 25.2 | 0.00 |
| NaH (31) | 0.98 | 0.9 | THF (DN = 20) | 4 | 0^{(c)} at a.t. | 67 | 1.5 | 59.5 | 7.85 | 88.81 |
| KHMDZ (27) | 1.8 | 0.6 | THF | 4 | 0^{(c)} at a.t. | 47.5 | 0 | 42 | 0 | 88.42 |
| MeONa (15) | 2.24 | 1 | Diisopropyl ether | 4 | 0^{(b)} at a.t. | 96 | 3.7 | traces | 23.3 | 2.60 |
| MeONa | 2.80 | 0.8 | Diisopropyl ether | 4 | 0^{(b)} at a.t. | 84.8 | n.d.^{(d)} | n.d.^{(d)} | 56.8 | n.d. |
| MeONa | 1.54 | 1.4 | MeOH | 4 | 0^{(c)} at a.t. | 100 | 10.2 | 0 | 60.6 | 0.00 |

^{(a)}¹⁹F NMR with internal standard;
^{(b)}addition at 0° C. and then left to react at a.t.;
^{(c)}addition at 0° C., left in the ice bath for ½ hour and then left to react at a.t.;
^{(d)}(16) and (21) could not be separated by ¹⁹F NMR: (16) + (21) = 12.3%

### EXAMPLE 2

### Dehydrohalogenation of the Precursor CF₃CH₂CHClCH₂Oac in the Presence of DMF

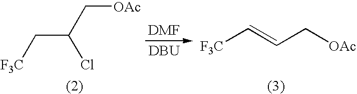

| Charges | RY (isolated) % |
|---|---|
| (2): 1830 g [8.95 mol] | 80% |
| DBU: 1500 g [9.85 mol] | |
| DMF: 7.5 liters | |
| 25° C., 2 to 3 hours | |

### EXAMPLE 3

### Dehydrohalogenation of the Precursor CF₃CH₂CHClCH₂OAc Using Potassium Carbonate

It was demonstrated that potassium carbonate makes it possible to carry out this dehydrochlorination reaction no longer at 25° C. but at 60° C. and in dipolar aprotic solvents, such as DMF or NMP (DN=27.3): 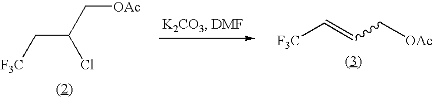
Effect of the Concentration:

(DMF, K₂CO₃ (5 eq.), 60° C., 4 h)

| % by weight^{(a)} | DC^{(b)} (2)% | RY^{(b)} (3)% |
|---|---|---|
| 11% | 100 | 92% (E + Z) |
| 16.5% | 100 | 85.5% (E + Z) |

^{(a)}Weight of (2)/weight of DMF;
^{(b)}Quantitative determination by ¹⁹F NMR with internal standard.

Effect of the Amount of K₂CO₃:

| K₂CO₃ (nbe eq.) | DC^{(a)} (2) (mol %) | RY^{(a)} (3) (mol %) |
|---|---|---|
| 5 | 100 | 88 + 10 |
| 3 | 100 | 86 + 9 |
| 1.5 | 99 | 87 + 9 |

^{(a)}Quantitative determination by ¹⁹F NMR with PhOCF₃ as internal standard.

Operating conditions: 60° C., 5 h, 3 g (15 mmol) of (2) per 28.5 g of DMF, i.e., depending on the K₂CO₃ stoichiometry, between 7 and 8.7% by weight.

### EXAMPLE 4

### Dehydrohalogenation of the Precursor CF₃CH₂CHBrCH₂OAc

The dehydrobromination reaction is carried out without any problems according to the same principle:

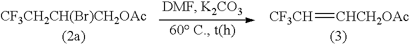

| Time^{(a)} | (2a)^{(b)} | (3E)^{(b)} | (3Z)^{(b)} |
|---|---|---|---|
| 45 min | 52.2 | 43 | 4.8 |
| 2 h | 31.4 | 61.7 | 6.9 |
| 3 h | 18.7 | 72.9 | 8.2 |
| 4 h | 12.5 | 78.5 | 8.9 |
| 5 h | 7.8 | 82.9 | 9.3 |
| 6.5 h | 2.4 | 87.6 | 9.9 |

(a) 02JGR910 test: Test in a jacketed 100 ml RPAS; stirring: 4 inclined blades (600 rev/min); (2a) (10 g at 96% by weight, 38.5 mmol), DMF (35 g), K₂CO₃ (18 g, 0.130 mol), (b) % area GC.

The reaction medium is left overnight at ambient temperature before treatment. After filtering (sintered glass funnel No. 4) and washing the cake with 10 ml of DMF, the filtrate is run onto 20 ml of water and this aqueous phase is extracted with MTBE (3×25 ml). The organic layer is quantitatively determined by ¹⁹F NMR with an internal standard:

| | CF₃CH═CHCH₂OAc | CF₃CH═CHCH₂OAc |
|---|---|---|
| CF₃CH₂CHBrCH₂OAc | (E) | (Z) |
| DC = 100% | RY = 74.5% | RY = 8% |

### EXAMPLE 5

### Cycloaddition Involving CF₃CH═CHCH₂OAc

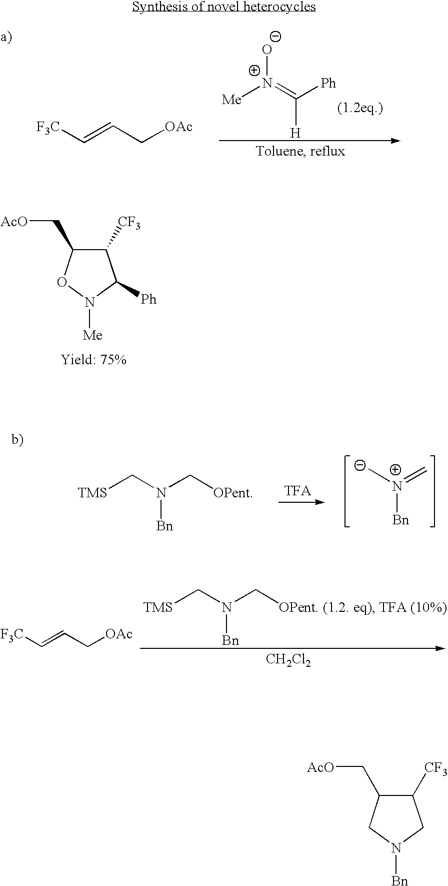

### EXAMPLE 6

### Dehydrochlorination with DBU

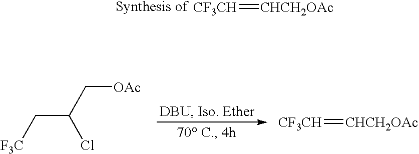

2-Chloro-4,4,4-trifluorobutyl acetate (3a) (12 g, 58.9 mmol) and diisopropyl ether (135 ml, 0.95 mol) are charged to a 250 ml three-necked flask equipped with a reflux condenser, a thermometer, a dropping funnel and a magnetic stirrer. 1,8-Diazabicyclo[5.4.0]undec-7-ene (8.98 g, 59 mmol) is added dropwise over 25 minutes. The reaction medium is heated at 70° C. for 4 hours. The progression of the reaction is monitored by GC injections. At the end of the reaction, a yield of product formed of 77.5% is determined by GC quantitative determination with an internal standard.

The reaction medium is run into 200 ml of water. Extraction is carried out with 3 times 100 ml of diisopropyl ether and the organic phases, combined beforehand, are dried over magnesium sulfate. After concentrating the diisopropyl ether at atmospheric pressure, 7 g (Yield: 71%) of the trifluorobutenyl acetate are isolated by conventional distillation.

Example of Dehydrobromination with K₂CO₃

Anhydrous NMP (10.25 g), potassium carbonate (5.13 g, 37.15 mmol) and then CF₃CH₂CHBrCH₂OAc (9.13 g (78% by weight), 28.6 mmol (1 eq.)) are introduced into a 100 ml jacketed reactor having a mechanical stirrer. The reaction medium is brought to 60° C. for 4-6 h. The progression of the reaction is monitored by GC (DC=100%, RY=95%). At the end of the reaction, the trifluorobutenyl acetate is distilled directly from the reaction medium. B.p.=45° C. under 30 mbar.

Structural Characterization:

Characteristic absorbances of the IR spectrum:
1750 cm⁻¹ νC═O and 1230 cm⁻¹ νC—O acetate functional group
1690 cm⁻¹ νC═C and δC—H (out of plane) unsaturation of the trans type
1127 cm⁻¹ νC—F

Chemical shifts of the ¹H, ¹⁹F and ¹³C resonance lines (in CDCl₃):

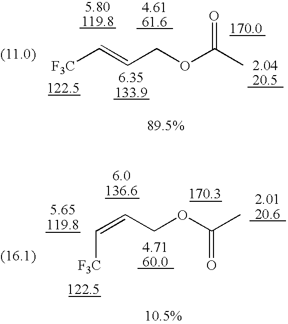

¹H Chemical shifts

( ) ¹⁹F Chemical shifts

¹³C Chemical shifts

### EXAMPLE 7

### Upgrading of CF₃CH═CHCH₂OAc

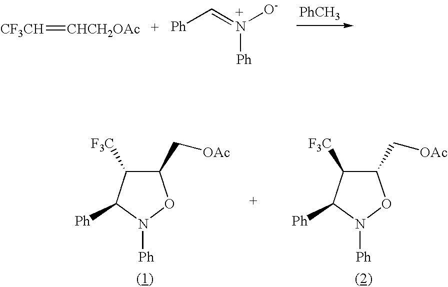

213 mg (1.27 mmol) of 4,4,4-trifluorobutenyl acetate (93/7 E/Z, 100% by weight), 3 g of toluene and 228 mg (1.12 mmol) of N,α-diphenyl nitrone (97% by weight) are added successively at ambient temperature to a 10 ml round-bottomed flask equipped with a reflux condenser. The solution is brought to reflux of the solvent (the nitrone is then rapidly dissolved) for 26 h. The toluene and the excess trifluorobutenyl acetate are evaporated under reduced pressure and the product, the mixture of stereoisomers, is obtained with a yield of 87% (ratio: (1)/(2), 25/1).

1-24. (canceled)

25. A compound of formula (I): 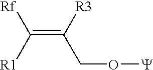
wherein:
R_{f} is a radical carrying a perfluoromethylene group, which group provides the link with the remainder of the molecule;
R₁ and R₃, which are the same or different, represent hydrogen, alkyl or aryl radicals; and
Ψ is an electron-withdrawing group such that Ψ-O—H is an acid with a pKa value in water of at most 8, optionally of at most 5.

26. The compound as claimed in claim 24, wherein Ψ is an electron-withdrawing group such that Ψ-O—H is an acid with a pKa value (in water) of at least 1, optionally of at least 2.

27. The compound as claimed in claim 25, wherein at least one of the R₁ and R₃ groups is a light alkyl of at most 4 carbons or a hydrogen.

28. The compound as claimed in claim 25, wherein the R_{f} radical is of formula (II):
EWG—(CX₂)ₚ—
wherein:
the X units, which are identical or different, represent a chlorine, a fluorine or a radical of the formula CₙF₂ₙ₊₁ with n an integer at most equal to 5, optionally to 2, with the proviso that the X units of the methylene group carrying the open bond are not chlorines and that at least one of them is a fluorine;
EWG is a hydrocarbon or electron-withdrawing group with an Hammett constant σₚ being >0, optionally at least equal to 0.2, which is inert and, when p is equal to 1, is an electron-withdrawing group; and p is a positive integer.

29. The compound as claimed in claim 25, wherein R₁ is hydrogen.

30. The compound as claimed in claim 25, wherein R₃ is hydrogen.

31. The compound as claimed in claim 25, wherein R₁ and R₃ are hydrogen.

32. The compound as claimed in claim 25, wherein R_{f} is a perfluoroalkyl of general formula CᵣF₂ᵣ₊₁ where r is a positive integer ranging from 1 to 10, optionally from 1 to 3.

33. The compound as claimed in claim 25, wherein R_{f} is trifluoromethyl, pentafluoroethyl or heptafluoropropyl.

34. The compound as claimed in claim 25, wherein Ψ is an acyl.

35. The compound as claimed in claim 25, wherein Ψ is an acyl such that the pKa value of Ψ-O—H is at least equal to 2 and optionally Ψ-O—H is an alkanoic acid with from 1 to 8 carbon atoms.

36. A process for the synthesis of a compound of formula (I) of the formula: 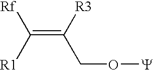
wherein:
R_{f} is a radical carrying a perfluoromethylene group, which group provides the link with the remainder of the molecule;
R₁ and R₃, which are the same or different, represent hydrogen, alkyl or aryl radicals; and
Ψ is an electron-withdrawing group such that Ψ-O—H is an acid with a pKa value in water of at most 8, optionally of at most 5 said process comprising the steps of:
a) reacting a compound of formula (III): 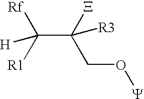
where Ξ is a leaving group chosen from pseudohalogens and halogens, with a strong nitrogenous base, the associated acid of which exhibits a pKa value of at least 12, and/or an anionic base, with the proviso that, when the base is a nonnitrogenous anionic base, the latter is in the presence of a polar solvent or a mixture of polar solvents.

37. The process as claimed in claim 36, wherein the base is a nonnitrogenous anionic base and said polar solvent exhibits a solvent with a donor number of at least 10, optionally of at least 20.

38. The process as claimed in claim 36, wherein the base is a nonnitrogenous anionic base and said polar solvent is a solvent which is miscible with water in any proportion.

39. The process as claimed in claim 36, wherein the base is a nonnitrogenous anionic base and in said polar solvent does not exhibit an acid functional group, that is to say that the pKa value of the most acidic hydrogen of said solvent is at least equal to 20, optionally to 30.

40. The process as claimed in claim 36, wherein said base is a nitrogenous anionic base being an alkali metal or an alkaline earth metal salt, of silylated amines and silylamines.

41. The process as claimed in claim 40, wherein said base is the anion of a silylamine of an alkali metal and alkaline earth metal salt of HMDZ (hexamethyldisilazane).

42. The process as claimed in claim 36, wherein said base is in the presence of a polar solvent, optionally a polar aprotic solvent.

43. The process as claimed in claim 36, wherein said base is a base carrying at least 2 trivalent nitrogens.

44. The process as claimed in claim 36, wherein said base carrying at least 2 trivalent nitrogens is such that said 2 nitrogens are conjugated via at least one double bond.

45. The process as claimed in claim 44, wherein said base carrying at least 2 trivalent nitrogens is such that said 2 trivalent nitrogens form a bonding system comprising an imine conjugated with the doublet of an amine.

46. A process for the synthesis of a nitrogenous heterocycle, comprising the step of carrying out a cyclocondensation of a compound of formula (I) as defined in claim 25 as precursor of a heterocycle substituted by an R_{f} group, with a cosubstrate carrying 2 double bonds.

47. The process as claimed in claim 46, wherein said cyclocondensation is of 3+2 type.

48. The process as claimed in claim 47, wherein the cosubstrate is an organic compound carrying a pentavalent nitrogen, at least one double bond of which connects said nitrogen to a carbon.
